(19) United States
(12) Patent Application Publication
(10) Pub. No.: US 2024/0050368 A1
(43) Pub. Date: Feb. 15, 2024

(54) ORAL ACTIVE AGENT DELIVERY SYSTEM FOR ANIMALS AND METHOD

(71) Applicants: Patrick Hunter Young, Bend, OR (US); Jeffrey Breit, Bend, OR (US)

(72) Inventors: Patrick Hunter Young, Bend, OR (US); Jeffrey Breit, Bend, OR (US)

(21) Appl. No.: 18/233,659

(22) Filed: Aug. 14, 2023

Related U.S. Application Data
(60) Provisional application No. 63/474,445, filed on Aug. 14, 2022.
(60) Provisional application No. 63/474,447, filed on Aug. 14, 2022.
(60) Provisional application No. 63/426,599, filed on Nov. 18, 2022.

(51) Int. Cl. A61K 9/00; A61K 38/03

(52) U.S. Cl. CPC A61K 9/006; A61K 38/03

(57) ABSTRACT

An oral active agent delivery system for animals, including horses, that includes a flexible orally dissolving film (ODF) for delivery of an active agent (i.e., active ingredient) to the oral cavity. The ODF may be applied directly to an oral mucosal surface or be laminated onto a bit that, in turn, is placed in the oral cavity of, for example, a horse.

### CROSS-REFERENCE TO RELATED APPLICATIONS

The present application claims the benefit of U.S. Provisional Patent Application Ser. Nos. 63/474,445 and 63/474,447, filed Aug. 14, 2022, and U.S. Provisional Patent Application Ser. No. 63/426,599, filed Nov. 18, 2022, each of which is incorporated herein by reference in its entirety.

### BACKGROUND

Giving an animal medicaments, nutrients, or other active agents (i.e., active ingredients), especially a large animal, such as an equus animal (e.g., horse), and ruminant animals, such as cattle/bovine, sheep/ovine, goats/caprine, and camelids, can be difficult. The animals do not understand that the active agents will help them. Traditionally, animals are treated similarly to humans, including the administration of pills and injections.

In addition, it is typically desired that after administration of an active agent, the agent is rapidly delivered to the circulation or bloodstream. Once in the bloodstream or circulation, active agents are typically metabolized by the animal. For example, if glucose or other sugars are administered, the animal quickly metabolizes them, and these energy sources are no longer available over time. That is, the active agent is used up before the need is exhausted, such as during a period of training.

Animals that are exercising have an ongoing need, for example, for metabolites and electrolytes to allow efficient metabolism. For example, when exercising a horse, there is an ongoing need to deliver nutrients while exercising in order for the animal to have continuous access to these nutrients.

Currently, there is a need for an efficient method to deliver metabolites for absorption into animals. Specifically, there is need for sublingual or mucosal delivery of active agents such as proteins, peptides, amino acids, electrolytes, and sugars. Oral delivery of energy metabolites during and immediately post exercise is required in animals in order to maintain physical health, aid in muscle recovery, aid in efficient exercise and at peak performance, and enhance next day performance.

Equus animals, such as horses, are typically provided an oral mixture of dextrose, amino acids, and salts post exercise in order to prevent the negative metabolic impacts of strenuous activity. The oral mixture is provided as a solution consumed by the animal and absorbed via the gastrointestinal (GI) tract. Alternatively, animals are fed a supplement or a nutrient pellet to aid in muscle recovery. This suffers from the same flaw as oral solutions as the animal is required to digest the supplement or nutrient via the GI tract.

What is needed is an oral active agent delivery system for animals, particularly equus animals, including horses, that includes a flexible orally dissolving film (ODF) for delivery of an active agent (i.e., active ingredient) to the oral cavity (e.g., mucosa) over a period of time.

### SUMMARY

The present disclosure provides an oral active agent delivery system for animals, particularly equus animals, preferably horses, ruminant animals such as cattle/bovine, sheep/ovine, goats/caprine, and camelids, as well as domesticated animals such as dogs/canine and cats/feline. The oral active agent delivery system includes a flexible orally dissolving film (ODF) (also often referred to as an oral thin film (OTF)) for delivery of an active agent (i.e., active ingredient) to the oral cavity (e.g., mucosa) over a period of time. The ODF may be applied directly to a mucosal surface (e.g., gum or other mucous membrane) or it may be laminated onto a bit that, in turn, is placed in the oral cavity of, for example, a horse. Although horses and other equus animals are preferred animals for application of the oral active agent delivery system described herein, other animals include ruminant animals such as cattle/bovine, sheep/ovine, goats/caprine, and camelids, and domesticated animals such as dogs/canine and cats/feline. Thus, herein, “animal” refers to a non-human animal.

The ODF dissolves and the active agent is delivered to the oral cavity and ultimately to the circulatory system (e.g., bloodstream) of the animal. Preferably, the ODF of the present disclosure does not dissolve immediately upon presentation to the oral mucosa. Instead, preferably, the ODF dissolves over a period of time, such as at least 5 minutes, and often up to 120 minutes.

In one embodiment, there is a laminated bit, preferably for use in an equus animal, comprising a bit and a flexible orally dissolving film (ODF) laminated onto the bit, wherein the ODF comprises one or more active agents.

In another embodiment, there is an oral active agent delivery system, preferaby for use with equus animals, comprising a flexible orally dissolving film (ODF) and one or more active agents, wherein when the delivery system is placed in an aqueous environment, at least 95% by weight of the ODF dissolves over a time period of 5 to 120 minutes.

Methods are also provided. In one embodiment, there is a method for delivering an active agent to an animal, preferably an equus animal. In another embodiment, there is a method for increasing the water consumption of an animal, preferably an equus animal. The methods include: selecting a flexible orally dissolving film (ODF) comprising an active agent; and applying the ODF to a surface within the oral cavity of the animal.

The terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

Herein, the term “comprises” and variations thereof do not have a limiting meaning where these terms appear in the description and embodiments. Such terms will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof).

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a,” “an,” and “the” are not intended to refer to only a singular entity, but include the general class of which a specific example may be used for illustration. The terms “a,” “an,” and “the” are used interchangeably with the term “at least one.” The phrases “at least one of” and “comprises at least one of” followed by a list refers to any of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.) and any sub-ranges (e.g., 1 to 5 includes 1 to 4, 1 to 3, 2 to 4, etc.).

The term “in the range” or “within a range” (and similar statements) includes the endpoints of the stated range.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular embodiments, including features, configurations, compositions, or characteristics may be combined in any suitable manner in one or more embodiments.

The above summary of the present disclosure is not intended to describe each disclosed embodiment or every implementation of the present disclosure. The description and drawings that follow more particularly exemplify illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the embodiments, and the equivalents of those structures. Any of the elements that are positively recited in this specification as alternatives may be explicitly included in the embodiments or excluded from the embodiments, in any combination as desired. Although various theories and possible mechanisms may have been discussed herein, in no event should such discussions serve to limit the claimable subject matter.

### BRIEF DESCRIPTION OF THE FIGURES

FIG. 1 is a diagram of an orally dissolving film shown in place in the oral cavity of a horse.

FIG. 2 is a diagram of laminated including an orally dissolving film for use in the oral cavity of an equus animal (e.g., a horse).

### DETAILED DESCRIPTION OF THE DISCLOSURE

The present disclosure provides an oral active agent delivery system for animals that includes a flexible orally dissolving film (ODF) for delivery of an active agent (i.e., active ingredient) to the oral cavity (e.g., mucosa) of the animals over a period of time. In one embodiment, an oral active agent delivery system for animals includes a flexible orally dissolving film (ODF) and one or more active agents, which may be applied directly to a mucosal surface (e.g., gum or other mucous membrane) of the animal. In another embodiment, the ODF is laminated onto a bit (i.e., that portion of a device used to connect reins to the mouth of the animal for the purpose of controlling the animal) that, in turn, is placed in the oral cavity of the animal.

The process of the present disclosure involves selecting an ODE containing an active agent and placing the resultant combination on a mucous membrane in the oral cavity. The hydrophilic nature of the ODF causes the film to stick (i.e., adhere) to the mucous membrane. As the film dissolves, the active agent is released and absorbed through the mucous membrane, and ultimately delivered to the circulatory system. of the animal. Herein, “circulatory system” includes either or both of the bloodstream or other lymphatic networks present with an animal.

A major advantage of the administration of active agents via the delivery system described herein is that the active agents are delivered via mucous membrane delivery, which bypasses the GI track and first pass metabolism. Also, and advantageously, administration of active agents via the delivery system described herein allows for controlled release of the active agents.

Preferably, the ODF of the present disclosure does not dissolve immediately upon presentation to the oral mucosa. Instead, preferably, the ODF dissolves over a period of time, such as at least 5 minutes. In certain embodiments the dissolution time period is often up to 120 minutes, up to 100 minutes, up to 75 minutes, up to 60 minutes, up to 30 minutes, or up to 15 minutes.

The delivery systems and methods of the present disclosure may be used to deliver an active agent to any of a variety of equus animals, such as horses, donkeys, asses, and zebras, ruminant animals, such as cattle/bovine, sheep/ovine, goats/caprine, and camelids, and domesticated animals, such as dogs/canine and cats/feline. Preferably, the delivery systems and methods of the present disclosure may be used to deliver an active agent to any of a variety of equus animals, more preferably, to horses.

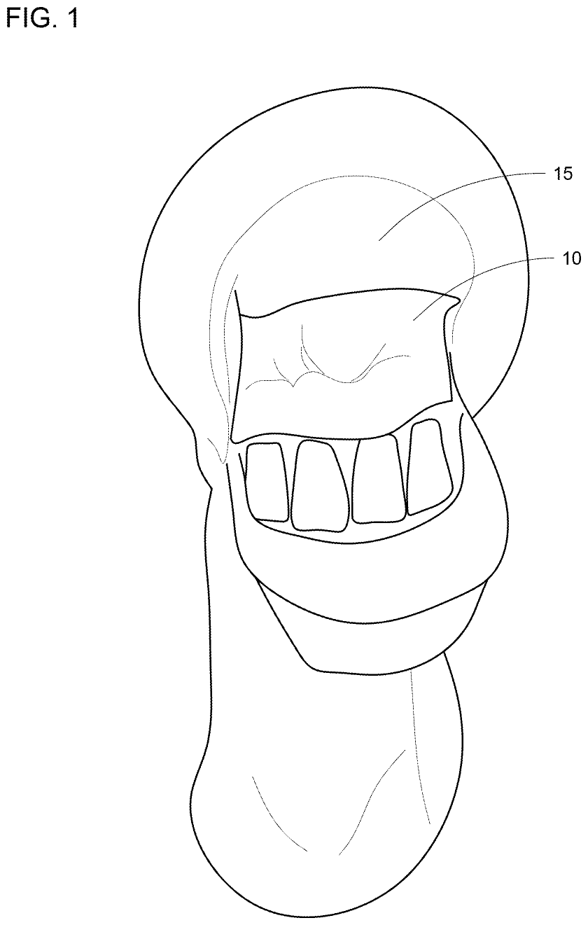

Flexible Orally Dissolving Films The active agent is contained in or on a sublingual gel in the form of a film, mucoadhesive film. strip, thin film strip, or patch, collectively referred to herein as oral thin films (OTFs) or orally dissolving films (ODFs). An example of an ODF 10 is shown in FIG. 1 adhered to the mucous membrane 15 in the oral cavity of a horse. Oral dissolving films provide an alternative delivery system of an active agent to that of capsules, pastes, pills, and tablets. They require no additional water, and maintain a relatively long shelf-life and freshness until consumed. They also provide relatively high bioavailability, particularly because they deliver the active agent(s) directly to the vasculature of an animal, preferably an equus animal, thus accelerating systemic delivery compared to GI delivery, and bypassing first pass metabolism.

ODFs are generally designed such that upon exposure to an aqueous environment, the ODF dissolves and this dissolution occurs rapidly, and often immediately. In the instant disclosure, however, the application of the ODF is such that it dissolves slowly over a course of time, preferably over a time of at least 5 minutes, and often up to as much as 2 hours. In certain embodiments, a preferred goal is that the ODF dissolves substantially completely within this period of time, leaving less than 5% w/w residue. That is, preferably at least 95% by weight of the ODF dissolves over a period of time of at least 5 minutes, and often over a period of time of up to 120 minutes.

The ODFs of the instant disclosure are designed to be flexible such that they are able to conform to a surface onto which they applied. In some embodiments, these flexible ODFs are able to be completed wrapped around an object, such as horse's bit.

In other embodiments, the ODFs may be any size and shape that is suitable for placement on the surface of a mucous surface of an animal, preferably an equus animal. For example, the ODFs may be of a planar shape, including by example, ovals, squares, rectangles, and are able to be contoured onto and be retained by a mucosal surface. Such mucosal surfaces may include those within the oral cavity of an animal, and may include the gums, any buccal surface or membrane, the gingiva, an interdental space, or the sublingual space.

In certain embodiments, the ODFs are in the general form of a rectangle or oval with a long axis being at least 3 inches in length and a short axis being at least 2 inches in length. In certain embodiments, the long is up to 5 inches in length and the short is up to 3 inches in length.

In certain embodiments, the ODFs are at least 0.05 millimeters thick. In certain embodiments, the ODFs are up to 0.1 millimeters thick.

There exist orally dissolving films that are used to deliver nutrients and medicaments to animals. See, for example, International Publication No. WO200518323A (Davidson). See also for example, U.S. Pat. No. 4,517,173 (Kizawa et al.), which discloses a film that adheres to mucous membrane. The polymers used in the compositions of such films may be suitable for use herein.

The polymers provide the structure and strength to the film. Suitable polymers for use in making the ODFs include a wide variety of hydrophilic or water-soluble polymers. For example, the polymer may include a mucoadhesive polymer (e.g., pullulan), a stabilizing polymer (e.g., a cellulose derivative (e.g., hydroxy propyl methyl cellulose), polyvinylpyrrolidone (PVP), polyvinyl alcohol (PVA)), a polysaccharide (e.g., maltodextrin or a heteropolysaccharide such as pectin), gelatin, or a combination thereof.

One or more polymers may be used to form the film in an amount of at least 30% by weight, based on the total weight of the ODF (polymer(s), active agent(s). and optional additive(s)). One or more polymers may be used to form the film in an amount of up to 60% by weight, based on the total weight of the ODF.

### Active Agents

Any of a wide variety of active agents that are typically delivered orally to an animal, preferably an equus animal, can be used in the systems and methods of the present disclosure.

The active agent may be one that generally promotes the health of the animal. The active agent may be one that promotes recovery from injury or ailment. The active agent may include a medication for treating a condition present in the animal or to prophylactically treat the animal.

Thus, the active agent may be a nutrient (i.e., a nutraceutical or nutritional aid such as a vitamin, hormone, herb, or an electrolyte) or a medicament (i.e., medication, drug, or pharmaceutical).

In order to achieve homogeneity and uniformity of the ODF, and produce a generally smooth film, the active agents are preferably selected to be soluble in water, alcohol (e.g., ethanol), or oral mucosa, at a temperature typically within a range of 35° C.-40° C. Also, in order to achieve an effective result, the active agents are preferably selected to be absorbed by the mucous membranes and oral mucosa.

The medicament may be an antibiotic, an anti-inflammatory, an anti-convulsant, an anesthetic, a flea repellant, a heartworm preventative, an anti-allergy drug, a dewormer, a chemotherapeutic medication, a hormone replacement medication, or an antifungal), or a combination thereof.

Suitable examples of active agents may be in the form of a protein, a peptide, a sugar, an electrolyte (i.e., a salt), an amino acid, or a combination thereof.

Exemplary electrolytes include salts of sodium, potassium, magnesium, calcium, and a combination thereof (such as sodium chloride, potassium chloride, magnesium sulfate, and calcium phosphate).

Exemplary sugars include dextrose, glucose, and a combination thereof.

Exemplary amino acids include a branched chain amino acid and other non-essential amino acids selected from the group consisting of acetyl L-carnitine, L-carnitine tartrate, L-glycine, L-leucine, alpha-lipoic acid, leucine, isoleucine, valine, carnitine, selenocysteine, lysine, glycine, glutamine arginine, aspartate, arginine, and a combination thereof.

Specific examples of a medicament may include, by way of example, one or more of: antibiotics (such as those known as Amoxicillin Clindamycim, Clavamox, Tetracyclines, Baytril, Cephalexin, Metronidazole); anti-inflammatories (such as those known as Deramaxx, Rimadyl, Metacam, Prednisone): anti-convulsants (such as those known as Phenobarbital, Potassium Bromide. Valium); anti-allergy drug (such as those available under the tradenames PREDNISONE and ANTIHISTAMINES); dewormers (such as those known as Pyrantel Pamoate, Nemex, Strongid, Panacur, Droncit); chemotherapeutic medications; hormone replacement medications; antifungals; nonsteroidal anti-inflammatory agents (NSAIDs); corticosteroids; sedatives; analgesics; tranquilizers; muscle relaxants; vasodilators, milk whey protein, tryptophan, alpha-2 antogonists, anti-pyretic agents; thyroid medications; and metabolic syndrome medications.

Anesthetics may include include, but are not limited to: benzocane, butamben, dibucaine, lidocaine, oxybuprocaine, pramoxine, proparacaine, proxymetacaine, and tetracaine (also named amethocaine).

The active agents of the present disclosure are not limited to those specifically described herein. Generally, any water-soluble agent that is able to transverse an animal's mucous membranes to be delivered to an animal's circulatory system (e.g., bloodstream) may be used.

One or more active agents may be present in, or on, an ODF in an amount of at least 1% by weight, based on the total weight of the ODF (polymer(s), active agent(s). and optional additive(s)). One or more active agents may be present in, or on, an ODF in an amount of up to 60% by weight, or up to 30% by weight, based on the total weight of the ODF. The total mass of the combination is typically at least 250 milligrams (mg) of total solids, and can be up to 10 grams (g) of total solids.

### Optional Additives

The ODF may include one or more optional additives. Examples include a plasticizer, a permeation enhancer (e.g., sodium caprate), a chelating agent (e.g., EDTA), a coloring agent, a flavorant, a stabilizer, or combinations thereof.

In order to achieve homogeneity and uniformity of the ODF, and produce a generally smooth film, the optional additives are preferably selected to be soluble in water, alcohol (such as ethanol), or oral mucosa, at a temperature typically within a range of 35° C.-40° C.

In some embodiments, the OUP may include a coloring agent to provide a visual indication of when the ODF dissolves and also a visual indication of which of the oral mucosa has been treated for easier identification of a treated area. Coloring agents may include, for example, a substance that allows a practitioner to identify an area that has been treated by an active agent.

Coloring agents may include any benign coloring that is visible to a practitioner. Accordingly, coloring agents may include by way of non-limiting example, natural pigments derived from natural sources such as vegetables, minerals or animals. Including: annatto, beet extract, caramel, beta-carotene, and grape skin extract.

Other examples of coloring agents may include: Caramel coloring (E150), or other coloring made from caramelized sugar; Annatto (E160b), a reddish-orange dye or other coloring made from the seed of the achiote; Chlorophyllin (E140), a green dye or other coloring made from chlorella algae; Cochineal (E120), a red dye or other coloring derived from the cochineal insect, Dactylopius coccus; Betanin (E162) or other coloring extracted from beets; Turmeric or other coloring derived from curcuminoids E100; Saffron or other coloring derived from carotenoids, E160a; Paprika (E160c); Lycopene (E160d); Elderberry juice; Pandan a green coloring or other coloring derived Pandamus amaryllifolius; Butterfly pea a blue dye or other coloring derived from Clitoria ternatea.

Other coloring agents may include coloring approved under the Pure Food and Drug Act including, but not limited to: FD&C Blue No. 1—Brilliant Blue FCF, E133 (blue shade); FD&C Blue No. 2—Indigotine, E132 (indigo shade); FD&C Green No. 3—Fast Green FCF, E143 (turquoise shade); FD&C Red No. 40—Akira Red AC, E129 (red shade); FD&C Red No. 3—Erythrosine, E127 (pink shade, commonly used in glace cherries); FD&C Yellow No. 5—Tartrazine, E102 (yellow shade); and FD&C Yellow No. 6—Sunset Yellow FCF, E110 (orange shade). Combinations of coloring agents may be used if desired.

Suitable plasticizers include polyethylene glycol, glycerin, propylene glygol. Combinations of plasticizers may be used if desired.

If use, one or more optional additives may be present in, or on, an ODF in an amount of at least 25% by weight, based on the total weight of the ODF (polymer(s), active agent(s). and optional additive(s)). One or more optional additives may be present in, or on, an ODF in an amount of up to 50% by weight, based on the total weight of the ODF.

### Laminated Bits

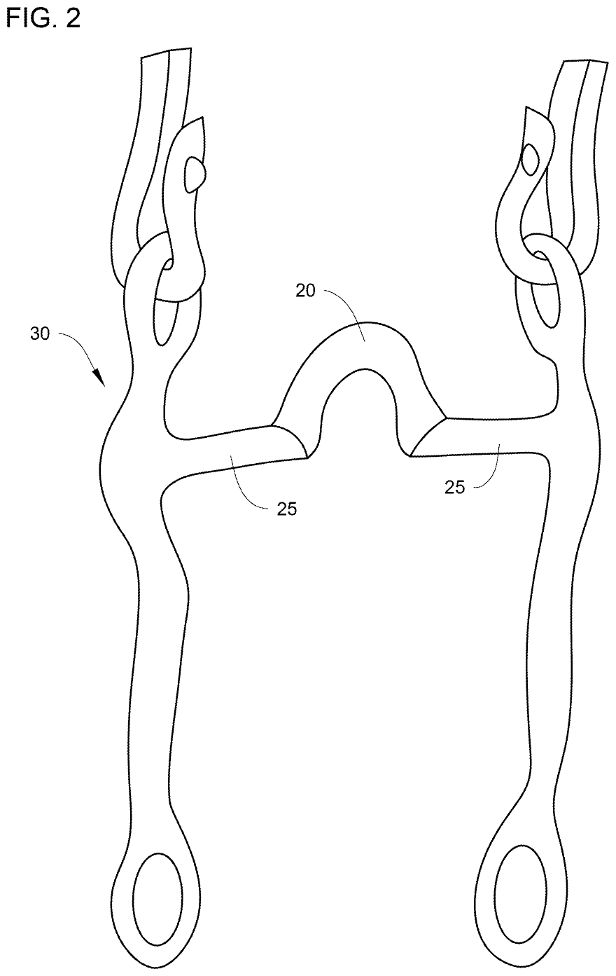

This disclosure provides a delivery system that includes an orally dissolving film 20 linked to an animal bridle, such as laminated to the bit 25 of the bridle 30, as shown in FIG. 2, A bridle is the general term used to describe the headgear by which an animal, such as a horse or other burden-bearing or pulling animal is governed. A bridle includes a bit, headstall, and reigns. A bit is that portion of a bridle that is designed to sit on an area in an animal's mouth that lacks teeth and is used to control an animal's movement. The bit is most commonly made of metals and synthetic materials. There are numerous types of bits such as those described in U.S. Pat. No. 9,181,079.

The ODF described herein is typically laminated to the bit. In one embodiment, the ODF is simply wrapped around a bit thereby creating a laminated bit, which may be subsequently placed in an animal's mouth. The ODF might be slightly dampened prior to application to a bit so as to avoid dissolution but encourage stickiness (i.e., adhesion).

Following insertion of the bit into the oral cavity in normal fashion well known to those that work with animals, particularly equus animals such as horse, the ODF dissolves.

### Methods of Making and Use

The present disclosure provides various methods of use.

In one embodiment, a method for delivering an active agent to an animal, preferably, an equus animal, is provided. The method includes: selecting an orally dissolving film (ODF) comprising the active agent; and applying the ODF to a surface within the oral cavity of the animal.

In another embodiment, a method for increasing the water consumption of an animal, preferably an equus animal, is provided. The method includes: selecting an orally dissolving film (ODF) comprising an active agent; and applying the ODF to a surface within the oral cavity of the animal.

In certain embodiments of such methods, the surface within the oral cavity may include an animal's gums, buccal surface, gingiva, interdental space, and the sublingual space. The active agent may be any of those disclosed herein.

In certain embodiments of such methods, preferably at least 95% by weight of the ODF dissolves over a time period of 5 to 120 minutes. In even more preferred methods, the time period for dissolution of the ODF is 5 to 15 minutes.

The methods may include the direct application of the ODF as described herein to a surface within the oral cavity of an animal, preferably an equus animal. Alternatively, the methods may include contacting a surface within the oral cavity of an animal by placing a laminated bit in the oral cavity of the animal, preferably an equus animal.

One or more active agents and one or more optional additives may be disposed on a surface of, or incorporated within, the polymer(s) of the orally dissolving film (ODF). This can be accomplished using well-known manufacturing techniques for orally dissolving films.

For example, ODFs disclosed herein can be made by solvent casting. In an exemplary method of film production, all the ingredients are combined into a slurry before casting into a film. Special care is taken to promote content homogeneity and uniformity, dissolving one or more active agents in water and heating them to an elevated temperature (typically, within a range of 110° C.-200° C.) before mixing them with other optional additives to form a slurry. This multi-step process typically promotes content homogeneity and uniformity.

Once all the ingredients are mixed together to form a slurry, a vacuum is applied to the slurry to remove air pockets. This is then placed in a conventional film-making maching to form a film. This allows for the production of an oral film strip without excess air bubbles, producing a generally smooth, consistent, and uniform oral film strip. The slurry is then baked in an oven at a temperature of typically 70° C. to 80° C. for a time sufficient to form the desired consistency and hydration (around 10% humidity is ideal) of the resultant material. Conditions effective to produce such ODFs are readily determined by one of skill in the art. Once formed and baked, the material is cut into, e.g., 2×3 inch film strips and placed in air-tight foil packaging and sealed.

The complete disclosures of the patents, patent documents, and publications cited herein are incorporated by reference in their entirety as if each were individually incorporated. To the extent that there is any conflict or discrepancy between this specification as written and the disclosure in any document that is incorporated by reference herein, this specification as written will control. Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the embodiments set forth herein as follows.

What is claimed:

1. A laminated bit for use in an animal comprising a bit and a flexible orally dissolving film (ODF) laminated onto the bit, wherein the ODF comprises one or more active agents.

2. The laminated bit of claim 1, wherein when placed in a aqueous environment, at least 95% by weight of the ODF dissolves over a time period of 5 to 120 minutes.

3. The laminated bit of claim 2, wherein the time period for dissolution of the ODF is 5 to 15 minutes.

4. The laminated bit of claim 3, wherein the active agent is disposed on a surface of, or incorporated within, the orally dissolving film (ODF).

5. The laminated bit of claim 1, wherein the active agent is a nutrient or a or a medicament.

6. The laminated bit of claim 1, wherein the active agent comprises a protein, a peptide, a sugar, an electrolyte, an amino acid, or a combination thereof.

7. The laminated bit of claim 6, wherein the active agent comprises an electrolyte selected from the group consisting of salts of sodium, potassium, magnesium, calcium, and a combination thereof.

8. The laminated bit of claim 6, wherein the active agent comprises a sugar selected from the group consisting of dextrose, glucose, and a combination thereof.

9. The laminated bit of claim 6, wherein the active agent comprises an amino acid selected from the group consisting of a branched chain amino acid and other non-essential amino acids selected from the group consisting of acetyl L-carnitine, L-carnitine tartrate, L-glycine, L-leucine, alpha-lipoic acid, leucine, isoleucine, valine, carnitine, selenocysteine, lysine, glycine, glutamine arginine, aspartate, arginine, and a combination thereof.

10. The laminated bit of claim 6, wherein the active agent comprises an agent selected from the group consisting of a decapeptide and a growth hormone.

11. The laminated bit of claim 10, wherein the decapeptide is BPC-157.

12. The laminated bit of claim 1, wherein the ODF comprises a mucoadhesive polymer, a stabilizing polymer, a polysaccharide, gelatin, or a combination thereof.

13. The laminated bit of claim 1, wherein the ODF further comprises a plasticizer, a permeation enhancer, a chelating agent, a coloring agent, a flavorant, a stabilizer, or combinations thereof.

14. An oral active agent delivery system for animals comprising a flexible orally dissolving film (ODF) and one or more active agents, wherein when the delivery system is placed in an aqueous environment, at least 95% by weight of the ODF dissolves over a time period of 5 to 120 minutes.

15. The delivery system of claim 14, wherein the time period for dissolution is 5 to 15 minutes.

16. The delivery system of claim 14, wherein the active agent comprises a protein, a peptide, a sugar, an electrolyte, an amino acid, or a combination thereof.

17. The delivery system of claim 14, wherein the aqueous environment is the oral cavity of an equus animal.

18. The delivery system of claim 17, wherein the aqueous environment is selected from the group consisting of an equus animal's gums, buccal surface, gingiva, interdental space, and the sublingual space.

19. The delivery system of claim 14, wherein the active agent is disposed on a surface of, or incorporated within, the orally dissolving film (ODF).

20. The delivery system of claim 14, wherein the ODF is shaped to conform to the contours of the surface onto which it is placed.

21. The delivery system of claim 20, wherein the ODF is in the general form of a rectangle or oval with a long axis being 5 inches in length and a short axis being 3 inches in length.

22. A method for delivering an active agent to an animal, the method comprising:
selecting a flexible orally dissolving film (ODF) comprising the active agent; and
applying the ODF to a surface within the oral cavity of the animal.

23. The method of claim 22, wherein at least 95% by weight of the ODF dissolves over a time period of 5 to 120 minutes.

24. The method of claim 23, wherein the time period for dissolution of the ODF is 5 to 15 minutes.

25. A method for increasing the water consumption of an animal, the method comprising:
selecting a flexible orally dissolving film (ODF) comprising an active agent; and
applying the ODF to a surface within the oral cavity of the animal.
